SEQUENCE LISTING (2 sequences)

SEQ ID NO: 1 - DNA, 1143 bp, Homo sapiens
atggggaagaatcctgtgcgtcctccacgagctcttccacctgtgccaagtcaagatgatattccacttagtcgtcctaagaaaaagaagcccagaacaaaaaacacaccagcaagtgcttctttggaaggccttgctcagactgctggtcgaaggccctctgagggcaatgagccatcaactaaagaactcaaagagcacccagaggctcctgttcaaagaagacagaaaaagacaaggctacctcttgaattggagacttcctccacccaaaagaagtcatctagttcatctttattacgaaatgaaaatggtattgatgcggagccagctgaggaggcagttattcaaaaacctcggaggaagacaaagaaaacccagccagcagaattacagtatgccaatgagctaggagtagaagatgaagacataatcactgatgagcaaactactgtggaacagcagtctgtattcactgcacccactggcattagccagcctgtaggcaaagtatttgtggaaaaaagccggagattccaggctgctgatcgttcagagttgataaagaccacagaaaacatagatgtgtcaatggacgtgaagccttcctggaccaccagagatgtggcacttacagtgcaccgggctttcaggatgattgttctagcaggagatcagctatccaacctctcaaaccttctgcaacaatacaagaccctagcgtatccattccagagtcttctgtacttgcttttggctctgagtacaatttcagcttttgacaggattgactttgctaaaatatcagtagcaatccgaaattttttggccctggatcctacagctttagcatctttcttgtactttactgctcttatactatctctgagtcagcaaatgacaagtgacagaatccacctttacacaccttcttctgttaatggtagcctctgggaagcaggaattgaggaacagattctccagccatggattgtggtgaatctcgtggtggctcttctggttggattatcttggctatttttgtcttataggccaggcatggatcttagtgaagagttaatgttctcctcagaggtggaagaatatcctgataaagagaaagaaatcaaagcctcttcataa

SEQ ID NO: 2 - protein, 400 aa, Homo sapiens
MGKNPVRPPRALPPVPSQDDIPLSRPKKKKPRTKNTPASASLEGLAQTAGRRPSEGNEPSTKELKEHPEAPVQRRQKKTRLPLELETSSTEKKSSSSSLLRNENGIDAEPAEEAVIQKPRRKTKKTQPAELQYANELGVEDEDIITDEQTTVEQQSVFTAPTGISQPVGKVFVEKSRRFQAADDSELIKTTENIDVSMDVKPSWTTRDVALTVHRAFRMIGLFSHGFLAGCAVWNIVVIYVLAGDQLSNLSNLLQQYKTLAYPFQSLLYLLLALSTISAFDRIDFAKISVAIRNFLALDPTALASFLYFTALILSLSQQMTSDRIHLYTPSSVNGSLWEAGIEEQILQPWIVVNLVVALLVGLSWLFLSYRPGMDLSEELMFSSEVEEYPDKEKEIKASS

(19) United States
(12) Patent Application Publication
(10) Pub. No.: US 2005/0058999 A1
(43) Pub. Date: Mar. 17, 2005

(54) Gene involved in apoptosis regulation

(76) Inventors: Robert Amson, Paris (FR); Adam Telerman, Paris (FR); Brent Passer, Paris (FR); Jean-Pierre Roperch, Lommoye (FR)

Correspondence Address: JONES DAY, 51 Louisiana Aveue, N.W, WASHINGTON, DC, 20001-2113, US

(21) Appl. No.: 10/451,862

(22) PCT Filed: Dec. 26, 2001
(86) PCT No.: PCT/FR01/04199

(30) Foreign Application Priority Data
Dec. 26, 2000 (FR) 00/17028

(51) Int. Cl. C12Q 1/68; C07H 21/04; C12N 9/02

(52) U.S. Cl. 435/6; 435/69.1; 435/189; 435/320.1; 435/325; 536/23.2

(57) ABSTRACT

This invention encompasses sequences of nucleic acids and polypeptides that are involved in the regulation of tumor suppression and apoptosis and methods of using the sequences. Methods of identification for compounds that are effective in treating disorders such as cancer and neurodegenerative disease are also disclosed.

The present invention relates to a nucleic acid sequence involved in the regulation of apoptosis and of tumor reversion by the p53 pathway, and also to the protein encoded by this nucleic acid sequence.

Apoptosis, or cell death, is a complex phenomenon which is regulated by many proteins, including the p53 protein. This protein interacts with many other proteins, and its expression, which induces the phenomena of cell death and of tumor reversion, can be correlated with the induction or the suppression of the expression of other cellular genes.

The inventors of the present invention have thus demonstrated genes which are induced and activated during the cascade leading to tumor suppression and/or apoptosis (TSAP for “Tumor Suppressor Activated Pathway”), or genes which are suppressed (TSIP for “Tumor Suppressor Inhibited Pathway”). These genes were in particular the subject of patent applications WO 97/22695 and WO 00/08147.

It is important to be able to understand precisely the mechanisms of the p53 cascade, in order to be able to generate novel compounds having antitumor activity (which can in particular induce apoptosis or tumor suppression), or which can be used for the treatment of neurodegenerative diseases. In fact, the inventors of the present invention have demonstrated that presenilin 1 (PS1), the role of which had been suggested in Alzheimer's disease, is identical to the TSIP 2 protein described in application WO 97/22695. Thus, it is legitimate to search for medicinal products which can interfere in apoptosis, in order to reduce this phenomenon, and which might be used in neurodegenerative diseases.

The inventors of the present application have demonstrated the interaction of the TSIP 2 protein (presenilin 1, PS1) with a protein which has itself been shown to be repressed in a model of cell death. This protein (SEQ ID No. 2) was thus called TSIP4, and it is a subject of the present invention, as are the nucleic acid sequences which encode said protein. The TSIP4 gene will in particular be preferred, it being understood that the term “gene” may be intended to mean either the cDNA sequence or the genomic DNA sequence, with or without the regulatory elements.

Thus, a subject of the present invention is a purified or isolated nucleic acid, characterized in that it comprises a nucleic acid sequence chosen from the group of following sequences:
a) SEQ ID No. 1;
b) the sequence of a fragment of at least 15 consecutive nucleotides of a sequence chosen from SEQ ID No. 1;
c) a nucleic acid sequence exhibiting a percentage identity of at least 80%, after optimal alignment, with a sequence defined in a) or b);
d) a nucleic acid sequence which hybridizes, under high stringency conditions, with a nucleic acid sequence defined in a) or b);
e) the complementary sequence or the RNA sequence corresponding to a sequence as defined in a), b), c) or d).

The nucleic acid sequence according to the invention defined in c) exhibits a percentage identity of at least 80%, after optimal alignment, with a sequence as defined in a) or b) above, preferably 90%, more preferably 95%, most preferably 98%, or 99%.

The terms “nucleic acid”, “nucleic acid sequence”, “polynucleotide”, “oligonucleotide”, “polynucleotide sequence” and “nucleotide sequence”, terms which will be used indifferently in the present description, are intended to denote a precise series of nucleotides, which may or may not be modified, making it possible to define a fragment or region of a nucleic acid, possibly comprising unnatural nucleotides, and which may correspond equally to a double-stranded DNA, a single-stranded DNA and products of transcription of said DNAs. Thus, the nucleic acid sequences according to the invention also encompass PNAs (peptide nucleic acids), or the like.

It should be understood that the present invention does not concern the nucleotide sequences in their natural chromosomal environment, i.e. in the natural state. They are sequences which have been isolated and/or purified, i.e. they have been removed directly or indirectly, for example by copying, their environment having been at least partially modified. The nucleic acids obtained by chemical synthesis are thus also intended to be denoted.

For the purpose of the present invention, the term “percentage identity” between two nucleic acids or amino acid sequences is intended to denote a percentage of nucleotides or of amino acid residues which are identical between the two sequences to be compared, obtained after the best alignment, this percentage being purely statistical and the differences between the two sequences being distributed randomly and over their entire length. The term “best alignment” or “optimal alignment” is intended to denote the alignment for which the percentage identity determined as below is highest. The sequence comparisons between two nucleic acid or amino acid sequences are conventionally carried out by comparing these sequences after having optimally aligned them, said comparison being carried out by segment or by “window of comparison” so as to identify and compare local regions of sequence similarity. The optimal alignment of the sequences for the comparison can be carried out, besides manually, by means of the local homology algorithm of Smith and Waterman (1981), by means of the local homology algorithm of Neddleman and Wunsch (1970), by means of the similarity search method of Pearson and Lipman (1988), by means of computer programs using these algorithms (GAP, BESTFIT, BLAST P, BLAST N, FASTA and TFASTA in the Wisconsin Genetics Software Package, Genetics Computer Group, 575 Science Drive, Madison, Wis.). In order to obtain the optimal alignment, the BLAST program is preferably used, with the BLOSUM 62 matrix. The PAM or PAM250 matrices can also be used.

The percentage identity between two nucleic acid or amino acid sequences is determined by comparing these two optimally aligned sequences, the nucleic acid or amino acid sequence to be compared possibly comprising additions or deletions compared to the reference sequence for optimal alignment between these two sequences. The percentage identity is calculated by determining the number of identical positions for which the nucleotide or the amino acid residue is identical between the two sequences, dividing this number of identical positions by the total number of positions compared, and multiplying the result obtained by 100 so as to obtain the percentage identity between these two sequences.

The expression “nucleic acid sequences exhibiting percentage identity of at least 80%, preferably 90%, more preferably 98%, after optimal alignment with a reference sequence” is intended to denote the nucleic acid sequences which, compared to the reference nucleic acid sequence, exhibit certain modifications, such as in particular a deletion, a truncation, an extension, a chimeric fusion and/or a substitution, in particular of the point type, and the nucleic acid sequence of which exhibits at least 80%, preferably 90%, more preferably 98%, identity, after optimal alignment, with the reference nucleic acid sequence. They are preferably sequences whose complementary sequences are capable of hybridizing specifically with the sequences SEQ ID No. 1 of the invention. Preferably, the specific or high stringency hybridization conditions will be such that they ensure at least 80%, preferably 90%, more preferably 98%, identity, after optimal alignment, between one of the two sequences and the sequence complementary to the other.

Hybridization under high stringency conditions means that the conditions of temperature and of ionic strength are chosen such that they allow the hybridization between two complementary DNA fragments to be maintained. By way of illustration, high stringency conditions for the hybridization step for the purposes of defining the polynucleotide fragments described above are advantageously as follows.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution of 0.15 M NaCl+0.015 M sodium citrate), 50% of formamide, 7% of sodium dodecyl sulfate (SDS), 10× Denhardt's, 5% of dextran sulfate and 1% of salmon sperm DNA; (2) hybridization per se for 20 hours at a temperature which depends on the length of the probe (i.e.: 42° C. for a probe>100 nucleotides in length), followed by two washes of 20 minutes at 20° C. in 2×SSC+2% SDS, and 1 wash of 20 minutes at 20° C. in 0.1×SSC+0.1% SDS. The final wash is carried out in 0.1×SSC+0.1% SDS for 30 minutes at 60° C. for a probe>100 nucleotides in length. The high stringency hybridization conditions described above for a polynucleotide of defined length can be adjusted by those skilled in the art for longer or shorter oligonucleotides, according to the teaching of Sambrook et al., 1989.

Among the nucleic acid sequences exhibiting a percentage identity of at least 80%, preferably 90%, more preferably 98%, after optimal alignment, with the sequence according to the invention, preference is also given to the nucleic acid sequences which are variants of SEQ ID No. 1, or of its fragments, i.e. all of the nucleic acid sequences corresponding to allelic variants, i.e. individual variations of the sequence SEQ ID No. 1. These natural mutated sequences correspond to polymorphisms present in mammals, in particular in humans, and in particular to polymorphisms which may lead to the occurrence of a pathological condition, such as, for example, a cell degeneration. Preferably, the present invention relates to the variant nucleic acid sequences in which the mutations lead to a modification of the amino acid sequence of the polypeptide, or of its fragments, encoded by the normal sequence of SEQ ID No. 1.

The expression “variant nucleic acid sequence” is also intended to denote any RNA or cDNA resulting from a mutation and/or variation of a splice site of the genomic nucleic acid sequence, the cDNA of which has the sequence SEQ ID No. 1.

The invention preferably relates to a purified or isolated nucleic acid according to the present invention, characterized in that it comprises or consists of the sequence SEQ ID No. 1, the sequence complementary thereto, or the RNA sequence corresponding to SEQ ID No. 1.

The invention also relates to a purified or isolated nucleic acid, characterized in that it encodes a polypeptide having a continuous fragment of at least 100, more preferably 150, most preferably 200, amino acids of the protein SEQ ID No. 2.

The primers or probes, characterized in that they comprise a sequence of a nucleic acid according to the invention, are also part of the invention.

Thus, the present invention also relates to the primers or the probes according to the invention which may make it possible, in particular, to demonstrate or to discriminate the variant nucleic acid sequences, or to identify the genomic sequence of the genes the cDNA of which is represented by SEQ ID No. 1, using in particular an amplification method such as the PCR method, or a related method.

The invention also relates to the use of a nucleic acid sequence according to the invention as a probe or primer, for detecting, identifying, assaying or amplifying a nucleic acid sequence.

According to the invention, the polynucleotides which can be used as a probe or as a primer in methods for detecting, identifying, assaying or amplifying nucleic acid sequence are a minimum of 15 bases, preferably of 20 bases, or better still of 25 to 30 bases, in length.

The probes and primers according to the invention can be directly or indirectly labeled with a radioactive or nonradioactive compound using methods well known to those skilled in the art, in order to obtain a detectable and/or quantifiable signal.

The polynucleotide sequences according to the invention which are unlabeled can be used directly as a probe or primer.

The sequences are generally labeled in order to obtain sequences which can be used for many applications. The labeling of the primers or of the probes according to the invention is carried out with radioactive elements or with nonradioactive molecules.

Among the radioactive isotopes used, mention may be made of ³²P, 33P, ³⁵S, ³H or ¹²⁵I. The nonradioactive entities are selected from ligands such as biotin, avidin, streptavidin or digoxigenin, haptens, dyes, and luminescent agents such as radioluminescent, chemiluminescent, bioluminescent, fluorescent or phosphorescent agents.

The polynucleotides according to the invention can thus be used as a primer and/or probe in methods using in particular the PCR (polymerase chain reaction) technique (Rolfs et al., 1991). This technique requires choosing pairs of oligonucleotide primers framing the fragment which must be amplified. Reference may, for example, be made to the technique described in U.S. Pat. No. 4,683,202. The amplified fragments can be identified, for example after agarose or polyacrylamide gel electrophoresis, or after a chromatographic technique such as gel filtration or ion exchange chromatography, and then sequenced. The specificity of the amplification can be controlled using the nucleotide sequences of polynucleotides of the invention as primers, and plasmids containing these sequences, or else the derived amplification products, as matrices. The amplified nucleotide fragments can be used as reagents in hybridization reactions in order to demonstrate the presence, in a biological sample, of a target nucleic acid of sequence complementary to that of said amplified nucleotide fragments.

The invention is also directed toward the nucleic acids which can be obtained by amplification using primers according to the invention.

Other techniques amplifying the target nucleic acid can advantageously be employed as an alternative to PCR (PCR-like) using a pair of primers of nucleotide sequences according to the invention. The term “PCR-like” is intended to denote all the methods which use direct or indirect reproductions of nucleic acid sequences, or else in which the labeling systems have been amplified; these techniques are of course known. In general, they involve amplification of the DNA with a polymerase; when the sample of origin is an RNA, a reverse transcription should be carried out beforehand. A very large number of methods currently exist for this amplification, such as, for example, the SDA (Strand Displacement Amplification) technique (Walker et al., 1992), the TAS (Transcription-based Amplification System) technique described by Kwoh et al. (1989), the 3SR (Self-Sustained Sequence Replication) technique described by Guatelli et al. (1990), the NASBA (Nucleic Acid Sequence Based Amplification) technique described by Kievitis et al. (1991), the TMA (Transcription Mediated Amplification) technique, the LCR (Ligase Chain Reaction) technique described by Landegren et al. (1988), the RCR (Repair Chain Reaction) technique described by Segev (1992), the CPR (Cycling Probe Reaction) technique described by Duck et al. (1990), and the Q-beta-replicase amplification technique described by Miele et al. (1983). Some of these techniques have since been improved.

When the target polynucleotide to be detected is an mRNA, an enzyme of the reverse transcriptase type is advantageously used, prior to carrying out an amplification reaction using the primers according to the invention or to carrying out a method of detection using the probes of the invention, in order to obtain a cDNA from the mRNA contained in the biological sample. The cDNA obtained will then serve as a target for the primers or the probes used in the method of amplification or of detection according to the invention.

The probe hybridization technique can be carried out in various ways (Matthews et al., 1988). The most general method consists in immobilizing the nucleic acid extracted from the cells of various tissues or from cells in culture, on a support (such as nitrocellulose, nylon or polystyrene), and in incubating, under well-defined conditions, the immobilized target nucleic acid with the probe. After hybridization, the excess probe is removed and the hybrid molecules formed are detected by the appropriate method (measuring the radioactivity, the fluorescence or the enzyme activity associated with the probe).

According to another embodiment of the nucleic acid probes according to the invention, the latter can be used as capture probes. In this case, a probe termed “capture probe” is immobilized on a support and is used to capture, by specific hybridization, the target nucleic acid obtained from the biological sample to be tested, and the target nucleic acid is then detected by virtue of a second probe, termed “detection probe”, labeled with a readily detectable element.

Among the advantageous nucleic acid fragments, it is thus necessary to mention, in particular, antisense oligonucleotides, i.e. oligonucleotides the structure of which ensures, by hybridization with the target sequence, inhibition of the expression of the corresponding product. It is also necessary to mention sense oligonucleotides which, by interaction with proteins involved in regulating the expression of the corresponding product, will induce either inhibition or activation of this expression.

Such oligonucleotides can be used as therapeutic products and medicinal products for regulating the phenomena of apoptosis and of tumor suppression. The use of these sense or antisense sequences can also be implemented in vitro, to define novel means of modeling and of studying apoptosis.

The present invention also relates to an isolated polypeptide, characterized in that it comprises a polypeptide chosen from:
a) a polypeptide of sequence SEQ ID No. 2;
b) a polypeptide which is a variant of a polypeptide of sequence defined in a);
c) a polypeptide homologous to a polypeptide defined in a) or b), comprising at least 80% identity with said polypeptide of a);
d) a fragment of at least 15 consecutive amino acids of a polypeptide defined in a), b) or c);
e) a biologically active fragment of a polypeptide defined in a), b) or c).

For the purpose of the present invention, the term “polypeptide” is intended to denote proteins or peptides.

The term “biologically active fragment” is intended to mean a fragment having the same biological activity as the peptide fragment from which it is deduced, preferably within the same order of magnitude (to within a factor of 10). Thus, the examples show that the TSIP4 protein (SEQ ID No. 2) has a potential role in the phenomena of apoptosis. A biologically active fragment of the TSIP4 protein therefore consists of a polypeptide derived from SEQ ID No. 2, also having a role in apoptosis.

Preferably, a polypeptide according to the invention is a polypeptide consisting of the sequence SEQ ID No. 2 (corresponding to the protein encoded by the TSIP4 gene) or of a sequence having at least 80% identity with SEQ ID No. 2 after optimal alignment.

The sequence of the polypeptide exhibits a percentage identity of at least 80%, after optimal alignment, with the sequences SEQ ID No. 2, preferably 90 or 95%, more preferably 98%, or 99%.

The expression “polypeptide the amino acid sequence of which exhibits a percentage identity of at least 80%, preferably 90%, more preferably 98%, after optimal alignment, with a reference sequence” is intended to denote the polypeptides exhibiting certain modifications compared to the reference polypeptide, such as in particular one or more deletions and/or truncations, an extension, a chimeric fusion, and/or one or more substitutions.

Among the polypeptides the amino acid sequence of which exhibits a percentage identity of at least 80%, preferably 90%, more preferably 98%, after optimal alignment, with the sequence SEQ ID No. 2, or with one of its fragments, according to the invention, preference is given to the variant polypeptides encoded by the variant nucleic acid sequences as defined above, in particular the polypeptides the amino acid sequence of which exhibits at least one mutation corresponding in particular to a truncation, deletion, substitution and/or addition of at least one amino acid residue compared to the sequence SEQ ID No. 2 or with one of its fragments, more preferably the variant polypeptides exhibiting a mutation associated with a pathological condition, such as a cancer or a neurodegenerative disease.

The present invention also relates to the cloning and/or expression vectors comprising a nucleic acid or encoding a polypeptide according to the invention. Such a vector can also contain the elements required for the expression and, optionally, for the secretion of the polypeptide in/from a host cell. Such a host cell is also a subject of the invention.

The vectors characterized in that they comprise a promoter and/or regulator sequence according to the invention are also part of the invention.

Said vectors preferably comprise a promoter, translation initiation and termination signals, and also suitable regions for regulating transcription. It must be possible for them to be maintained stably in the cell and they can optionally have particular signals specifying the secretion of the translated protein.

These various control signals are chosen as a function of the cellular host used. To this effect, the nucleic acid sequences according to the invention can be inserted into vectors which replicate autonomously in the chosen host, or vectors which integrate in the chosen host.

Among the autonomously replicating systems, use is preferably made, depending on the host cell, of systems of the plasmid or viral type, the viral vectors possibly being in particular adenoviruses (Perricaudet et al., 1992), retroviruses, lentiviruses, poxviruses or herpes viruses (Epstein et al., 1992). Those skilled in the art are aware of the technology which can be used for each of these systems.

When integration of the sequence into the host cell's chromosomes is desired, use may, for example, be made of systems of the plasmid or viral type; such viruses are, for example, retroviruses (Temin, 1986) or AAVs (Carter, 1993).

Among the nonviral vectors, preference is given to naked polynucleotides such as naked DNA or naked RNA according to the technique developed by the company VICAL, bacterial artificial chromosomes (BACs), yeast artificial chromosomes (YACs) for expression in yeast, mouse artificial chromosomes (MACs) for expression in murine cells and, preferably, human artificial chromosomes (HACs) for expression in human cells.

Such vectors are prepared according to the methods commonly used by those skilled in the art, and the clones resulting therefrom can be introduced into a suitable host by standard methods, such as, for example, lipofection, electroporation, heat shock, transformation after chemical permeabilization of the membrane, or cell fusion.

The invention also comprises the host cells, in particular the eukaryotic or prokaryotic cells, transformed with the vectors according to the invention, and also the transgenic animals, preferably mammals, except humans, comprising one of said transformed cells according to the invention. These animals can be used as models, for studying the etiology of inflammatory and/or immune diseases, and in particular inflammatory diseases of the digestive tract, or for studying cancers.

Among the cells which can be used for the purposes of the present invention, mention may be made of bacterial cells (Olins and Lee, 1993), but also yeast cells (Buckholz, 1993), as well as animal cells, in particular mammalian cell cultures (Edwards and Aruffo, 1993), and especially chinese hamster ovary (CHO) cells. Mention may also be made of insect cells in which use may be made of methods using, for example, baculoviruses (Luckow, 1993). A preferred cellular host for expression of the proteins of the invention consists of COS cells.

Among the mammals according to the invention, preference is given to animals such as rodents, in particular mice, rats or rabbits, expressing a polypeptide according to the invention.

Among the mammals according to the invention, preference is also given to animals such as mice, rats or rabbits, characterized in that the gene encoding the protein of sequence SEQ ID No. 2, or the sequence of which is encoded by the homologous gene in these animals, is not functional, is knocked out, or exhibits at least one mutation.

These transgenic animals are obtained, for example, by homologous recombination on embryonic stem cells, transfer of these stem cells to embryos, selection of the chimeras affected in the reproductive lines, and growth of said chimeras.

The transgenic animals according to the invention can thus overexpress the gene encoding the protein according to the invention, or their homologous gene, or express said gene into which a mutation is introduced. These transgenic animals, in particular mice, are obtained, for example, by transfection of the copy of this gene under the control of a strong promoter which is ubiquitous in nature, or selective for a tissue type, or after viral transcription.

Alternatively, the transgenic animals according to the invention can be made deficient for the gene encoding the polypeptide of sequence SEQ ID No. 2, or its homologous gene, by inactivation using the LOXP/CRE recombinase system (Rohlmann et al., 1996) or any other system of inactivation of the expression of this gene.

The cells and mammals according to the invention can be used in a method for producing a polypeptide according to the invention, as described below, and can also be used as an analytical model.

The transformed cells or mammals as described above can also be used as models in order to study the interactions between the polypeptides according to the invention, and the chemical or protein compounds involved directly or indirectly in the activities of the polypeptides according to the invention, in order to study the various mechanisms and interactions which come into play.

They can in particular be used to select products which interact with the polypeptides according to the invention, in particular the protein of sequence SEQ ID No. 2 or its variants according to the invention, as a cofactor or as an inhibitor, in particular a competitive inhibitor, or else which have agonist or antagonist activity with respect to the activity of the polypeptides according to the invention. Preferably, said transformed cells or transgenic animals are used as a model in particular for selecting products for combating pathological conditions associated with abnormal expression of this gene.

The invention also relates to the use of a cell, of a mammal or of a polypeptide according to the invention, for screening chemical or biochemical compounds which can interact directly or indirectly with the polypeptides according to the invention, and/or which are capable of modulating the expression or the activity of these polypeptides.

Similarly, the invention also relates to a method for screening compounds capable of interacting, in vitro or in vivo, with a nucleic acid according to the invention, using a nucleic acid, a cell or a mammal according to the invention, and detecting the formation of a complex between the candidate compounds and the nucleic acid according to the invention.

The compounds thus selected are also subjects of the invention.

Such a compound according to the invention may be a compound which has a chemical structure, a lipid, a sugar, a protein, a peptide, a protein-lipid, protein-sugar, peptide-lipid or peptide-sugar hybrid compound, or a protein or a peptide to which chemical branches have been added.

Among the chemical compounds envisaged, they may contain one or more rings, which may or may not be aromatic, and also several residues of any type (in particular lower alkyl, i.e. exhibiting between 1 and 6 carbon atoms).

The invention also relates to the use of a nucleic acid sequence according to the invention, for synthesizing recombinant polypeptides.

The method for producing a polypeptide according to the invention in recombinant form, which is itself included in the present invention, is characterized in that the transformed cells, in particular the mammalian cells of the present invention, are cultured under conditions which allow the expression of a recombinant polypeptide encoded by a nucleic acid sequence according to the invention, and in that said recombinant polypeptide is recovered.

The recombinant polypeptides, characterized in that they can be obtained using said method of production, are also part of the invention.

The recombinant polypeptides obtained as indicated above can be in both glycosylated and nonglycosylated form, and may or may not exhibit the natural tertiary structure.

The sequences of the recombinant polypeptides can also be modified in order to improve their solubility, in particular in aqueous solvents.

Such modifications are known to those skilled in the art, such as, for example, the deletion of hydrophobic domains or the substitution of hydrophobic amino acids with hydrophilic amino acids.

These polypeptides can be produced from the nucleic acid sequences defined above, according to the techniques of recombinant polypeptide production known to those skilled in the art. In this case, the nucleic acid sequence used is placed under the control of signals which allow its expression in a cellular host.

An effective system for producing a recombinant polypeptide requires having a vector and a host cell according to the invention.

These cells can be obtained by introducing into host cells a nucleotide sequence inserted into a vector as defined above, and then culturing said cells under conditions which allow the replication and/or the expression of the transfected nucleotide sequence.

The methods used to purify a recombinant polypeptide are known to those skilled in the art. The recombinant polypeptide can be purified from cell lysates and extracts, from the culture medium supernatant, by methods used individually or in combination, such as fractionation, chromatography methods, immunoaffinity techniques using specific monoclonal or polyclonal antibodies, etc.

The polypeptides according to the present invention can also be obtained by chemical synthesis using one of the many known peptide syntheses, for example the techniques using solid phases (see in particular Stewart et al., 1984) or techniques using partial solid phases, by fragment condensation or by conventional synthesis in solution.

The polypeptides which are obtained by chemical synthesis and which can comprise corresponding unatural amino acids are also included in the invention.

The mono- or polyclonal antibodies or their fragments, chimeric antibodies or immunoconjugated antibodies, characterized in that they are capable of specifically recognizing a polypeptide according to the invention, are part of the invention.

Specific polyclonal antibodies can be obtained from a serum of an animal immunized against the polypeptides according to the invention, in particular produced by genetic recombination or by peptide synthesis, according to the usual procedures.

The advantage of antibodies which specifically recognize certain polypeptides, or variants, or their immunogenic fragments, according to the invention is in particular noted.

The mono- or polyclonal antibodies or their fragments, chimeric antibodies or immunoconjugated antibodies, characterized in that they are capable of specifically recognizing the polypeptide of sequence SEQ ID No. 2, are particularly preferred.

The specific monoclonal antibodies can be obtained according to the conventional method of hybridoma culture described by Kohler and Milstein (1975).

The antibodies according to the invention are, for example, chimeric antibodies, humanized antibodies, or Fab or F(ab′)₂ fragments. They can also be in the form of immunoconjugates or of antibodies which are labeled in order to obtain a detectable and/or quantifiable signal.

The invention also relates to methods for detecting and/or purifying a polypeptide according to the invention, characterized in that they use an antibody according to the invention.

The invention also comprises purified polypeptides, characterized in that they are obtained using a method according to the invention.

Moreover, besides their use for purifying the polypeptides, the antibodies of the invention, in particular the monoclonal antibodies, can also be used for detecting these polypeptides in a biological sample.

They thus constitute a means of immunocytochemical or immunohistochemical analysis of the expression of the polypeptides according to the invention, in particular the polypeptide of sequence SEQ ID No. 2 or one of its variants, on specific tissue sections, for example by immunofluorescence or gold labeling, or with enzyme immunoconjugates.

They can in particular make it possible to demonstrate abnormal expression of these polypeptides in the tissues or biological specimens.

More generally, the antibodies of the invention can advantageously be used in any situation where the expression of a normal or mutated polypeptide according to the invention must be observed.

Thus, a method for detecting a polypeptide according to the invention in a biological sample, comprising the steps of bringing the biological sample into contact with an antibody according to the invention and demonstrating the antigen-antibody complex formed, is also a subject of the invention, as is a kit for implementing such a method. Such a kit contains in particular:
a) a monoclonal or polyclonal antibody according to the invention;
b) optionally, reagents for constituting a medium suitable for the immunoreaction;
c) the reagents for detecting the antigen-antibody complex produced during the immunoreaction.

The antibodies according to the invention can also be used in the treatment of a neurodegenerative disease or of a cancer, in humans, when abnormal expression of the TSIP4, PS1 or p53. gene, or a gene described by the inventors of the present application in the application WO 97/22695 or WO 00/08147, is observed. An abnormal expression means an overexpression, an underexpression or the expression of a mutated protein.

These antibodies can be obtained directly from human serum, or can be obtained from animals immunized with polypeptides according to the invention, and then “humanized”, and can be used as they are or in the preparation of a medicinal product intended for the treatment of the abovementioned diseases.

The methods for determining an allelic variability, a mutation, a deletion, a loss of heterozygosity, or any genetic abnormality of the gene according to the invention, characterized in that they use a nucleic acid sequence, a polypeptide or an antibody according to the invention, are also part of the invention.

It is possible to detect the mutations in the sequence of the TSIP4 gene suppressed during p53-induced apoptosis, directly by analysis of the nucleic acid and of the sequences according to the invention (genomic DNA, RNA or cDNA), but also by the polypeptides according to the invention. In particular, the use of an antibody according to the invention which recognizes an epitope carrying a mutation makes it possible to discriminate between a “healthy” protein and a protein “associated with a pathological condition”.

The precise description of the mutations which can be observed in the TSIP4 gene can thus make it possible to form the bases of a molecular diagnosis for neurodegenerative diseases and cancers, or any disease involving apoptosis. Such an approach, based on searching for mutations in the gene, will make it possible to contribute to the diagnosis of these diseases and, optionally, will make it possible to reduce the extent of certain additional, invasive or expensive, examinations. The invention forms the bases of such a molecular diagnosis based on the search for mutations in TSIP4.

In particular, preference is given to a method of diagnosis and/or of prognostic evaluation of a neurodegenerative disease or of a cancer, characterized in that the presence of at least one mutation and/or an alteration of expression of the gene corresponding to SEQ ID No. 1 is determined, using a biological specimen from a patient, by analyzing all or part of a nucleic acid sequence corresponding to said gene.

This method of diagnosis and/or of prognostic evaluation can be used preventatively (study of a predisposition to a neurodegenerative disease or to cancer), or to serve in establishing and/or confirming a clinical condition in a patient.

Preferably, the neurodegenerative disease is Alzheimer's disease.

The analysis can be carried out by sequencing all or part of the gene, or by other methods known to those skilled in the art. Methods based on PCR, for example PCR-SSCP, which makes it possible to detect point mutations, can in particular be used.

The analysis can also be carried out by attachment of a probe according to the invention, corresponding to one of the sequences SEQ ID No. 1, to a DNA chip and hybridization on these microplates. A DNA chip containing a sequence according to the invention is also one of the subjects of the invention.

Similarly, a protein chip containing an amino acid sequence according to the invention is also a subject of the invention. Such a protein chip makes it possible to study the interactions between the polypeptides according to the invention and other proteins or chemical compounds, and can thus be of use for screening compounds which interact with the polypeptides according to the invention. The protein chips according to the invention can also be used to detect the presence of antibodies directed against the polypeptides according to the invention, in the serum of patients. A protein chip containing an antibody according to the invention can also be used.

Those skilled in the art also know how to use techniques for studying the alteration of the expression of a gene, for example by studying the mRNA (in particular by Northern Blotting or by RT-PCR experiments, with probes or primers according to the invention), or the protein expressed, in particular by Western Blotting using antibodies according to the invention.

The invention also relates to methods for obtaining an allele of the TSIP4 gene, associated with a detectable phenotype, comprising the following steps:
a) obtaining a nucleic acid sample from an individual expressing said detectable phenotype;
b) bringing said nucleic acid sample into contact with an agent capable of specifically detecting a nucleic acid encoding the TSIP4 protein;
c) isolating said nucleic acid encoding the TSIP4 protein.

Such a method can be followed by a step of sequencing all or part of the nucleic acid encoding the TSIP4 protein, which makes it possible to predict the susceptibility to a neurodegenerative disease or to a cancer.

The agent capable of specifically detecting a nucleic acid encoding the TSIP4 protein is advantageously an oligonucleotide probe according to the invention, which can be made up of DNA, RNA or PNA, which may or may not be modified. The modifications may include radioactive or fluorescent labeling, or may be due to modifications in the bonds between the bases (phosphorothioates or methylphosphonates for example). Those skilled in the art are aware of the protocols for isolating a specific DNA sequence. Step b) of the method described above may also be an amplification step as described previously.

The invention also relates to a method for detecting and/or assaying a nucleic acid according to the invention in a biological sample, comprising the following steps of bringing a probe according to the invention into contact with a biological sample and detecting and/or assaying the hybrid formed between said polynucleotide and the nucleic acid of the biological sample.

Those skilled in the art know how to implement such a method, and can in particular use a reagent kit comprising:
a) a polynucleotide according to the invention, used as a probe;
b) the reagents required to carry out a hybridization reaction between said probe and the nucleic acid of the biological sample;
c) the reagents required for detecting and/or assaying the hybrid formed between said probe and the nucleic acid of the biological sample;
   which is also a subject of the invention.

Such a kit can also contain positive or negative controls in order to ensure the quality of the results obtained.

However, in order to detect and/or assay a nucleic acid according to the invention, those skilled in the art may also carry out an amplification step using primers chosen from the sequences according to the invention.

Finally, the invention also relates to the compounds chosen from a nucleic acid, a polynucleotide, a vector, a cell or an antibody according to the invention, or the compounds obtained using the screening methods according to the invention, as a medicinal product, in particular for preventing and/or treating a neurodegenerative disease or a cancer, associated with the presence of at least one mutation of the gene corresponding to SEQ ID No. 1, and/or with abnormal expression of the protein corresponding to SEQ ID No. 2.

The present invention also relates to methods for screening and identifying products which can interfere in the p53 cascade, and thus induce tumor reversion and/or apoptosis or, conversely, decrease the phenomena of apoptosis.

This aspect of the present invention is based on the fact that the TSIP4 protein, which is the subject of the present invention, binds to the TSIP2 protein described in patent application WO 97/22695, the role of which in Alzheimer's disease has been reported.

In a first embodiment, the invention is directed toward a method for screening, selecting or identifying a compound which interferes with, decreases or inhibits the binding of TSIP4 to TSIP2, having the steps of:
a) bringing said compound into contact with a system for determining, in vitro, the binding between TSIP4 and TSIP2;
b) identifying the decrease and/or the inhibition of the binding between TSIP4 and TSIP2.

A subject of the present invention is also in particular a method for screening, selecting or identifying a compound for decreasing and/or inhibiting tumor suppression and/or cell death (apoptosis), having the steps of:
a) bringing compounds into contact with a system for determining, in vitro, the binding between TSIP4 and TSIP2;
b) identifying the compounds which induce the decrease and/or the inhibition of the binding between TSIP4 and TSIP2;
c) studying the compounds identified in step b) in a model for measuring the phenomena of apoptosis and/or tumor suppression;
d) identifying the decrease and/or the inhibition in tumor suppression and/or cell death in said model compared to a control model with which said compound has not been brought into contact.

Some models for studying/measuring apoptosis and/or tumor suppression have already been described, and consist in particular of the in vitro models described by Tellerman et al. (1993, Proc. Natl. Acad. Sci. USA, 90, 8702-6), Amson et al. (1996, Proc. Natl. Acad. SCI; USA, 93, 3953-7) or Nemani et al. (1996, Proc. Natl. Acad. Sci. USA, 93, 9039-42). In order to determine compounds which make it possible to increase tumor suppression and/or cell death (apoptosis), it is also possible to combine various methods according to the invention; it is in particular possible to thus obtain a method for screening, selecting or identifying compounds whose function is an increase in tumor suppression and/or cell death (apoptosis), having the steps of:
a) bringing said compound into contact with a system for determining, in vitro, the binding between TSIP4 and TSIP2;
b) identifying the compounds which induce the decrease and/or the inhibition in the binding between TSIP4 and TSIP2;
c) bringing the compounds selected in step b) into contact in a model for measuring the phenomena of apoptosis and/or tumor suppression;
d) identifying the increase in tumor suppression and/or cell death in said model compared to a control model with which said compound has not been brought into contact.

Such a method is therefore also a subject of the present invention.

The present invention therefore uses the fact that the TSIP4 and TSIP2 proteins can bind to one another. It is therefore advantageous to identify the domains of each protein which are effectively in contact with the other protein. As a result, this should make it possible to be able to use the peptides thus identified as decoys or agonists for the complete proteins. This may thus make it possible to define compounds which will interfere in the TSIP4-TSIP2 binding, and which may possibly induce tumor suppression and/or apoptosis or, conversely, decrease these phenomena.

More generally, the present invention makes it possible to identify regions of TSIP4 involved in binding with the TSIP2 protein, using a method comprising the steps of:
a) bringing peptides derived from the TSIP4 protein into contact in a system for determining, in vitro, the binding between TSIP4 and TSIP2;
b) identifying the peptides which bring about the decrease in binding between TSIP4 and TSIP2 in said system.

It is obvious that the present invention also makes it possible to determine the regions of TSIP2 involved in the binding with TSIP4, according to methods similar to the methods described above, and that these regions can in particular be used as decoys when the intention is to decrease tumor suppression and/or apoptosis.

The term “region” of a protein is in particular intended to mean peptides of primary sequence derived from the primary sequence of said protein.

These regions thus identified can be tested, like the products described above, for their properties in the regulation of tumor suppression and/or apoptosis.

The present invention therefore makes it possible to identify products which make it possible to interact with the TSIP4-TSIP2 binding, and which therefore may be of value in the regulation of apoptosis and/or tumor suppression. However, it is possible that these products, in order to be able to be used for a therapeutic treatment, in particular cancer or neurodegenerative diseases, require optimization in order to have greater activity and/or less toxicity.

Specifically, the development of a medicinal product is often carried out according to the following principle:
screening compounds having a desired activity using a suitable method,
selecting the compounds which satisfy the “specifications”,
determining the structure (in particular the sequence (optionally tertiary sequence) if they are peptides, formula and backbone if they are chemical compounds) of the compounds selected,
optimizing the compounds selected, by modification of the structure (for example by changing the stereochemical conformation (for example switching from L to D for the amino acids in a peptide), adding substituents to the peptide or chemical backbones, in particular by grafting residues onto the backbone, modification of the peptides (see in particular Gante (“Peptidomimetics”, in Angewandte Chemie-International Edition Engl. 1994, 33. 1699-1720))),
passing and screening the compounds thus obtained on suitable models which are often models closer to the pathology studied. At this stage, animal models are in particular often used, in general in rodents (mice, rats, etc.) or in dogs, or even primates.

The animal models which can be used are, for example, for cancer, models based on immunodepressed mice (for example scid/scid), into which tumor cells, which will lead to the development of tumors, are injected (in particular subcutaneously). The effectiveness of the potentially antitumor compounds is studied, for example by measuring the size of the tumors formed.

To study neurodegenerative diseases, the model described by Amson et al. (2000, Proc. Natl. Acad. Sci. USA, 97, 5346-50), which consists of p53-deficient mice, or the model described in Chen et al., Janus et al., and Morgan et al. (2000, Nature, 408 pp. 975-985), can be used.

Thus, an object of the present invention is in particular to make it possible to identify compounds which might be used for the treatment of cancer, in that they have an activity of increasing tumor suppression and/or apoptosis. One of the subjects of the present invention is therefore a method comprising the steps of:
a) implementing a method according to the invention, for identifying compounds having a certain activity of increasing tumor suppression and/or apoptosis,
b) modifying the product selected in step a),
c) testing the product modified in step b) in in vitro and/or in vivo methods, on relevant models of tumor suppression and/or apoptosis,
d) identifying the product which makes it possible to obtain an activity of tumor suppression and/or apoptosis greater than the activity obtained for the product selected in step a).

Step d) can be replaced with a step d′), which would be:
d′) identifying the product which makes it possible to obtain the desired biological effect with less toxicity in an animal model (when one of the models used in step c) is in vivo).

When the tests are carried out on models of apoptosis or of tumor suppression in vitro, it is possible, for example, to use the model K256/KS described by Tellerman et al. (1993, Proc. Natl. Acad. Sci. USA, 90, 8702-6). It is also possible to use the M1-LTR cells described by Amson et al. (1996, Proc. Natl. Acad. SCI; USA, 93, 3953-7), or the U937/US3-US4 cells described by Nemani et al. (1996, Proc. Natl. Acad. Sci. USA, 93, 9039-42).

The in vivo trials can be carried out by injecting these cells into animals, in particular immunodepressed mice and studying the effects of the various compounds tested.

Those skilled in the art will be able to define the conditions and the thresholds necessary for identifying a product which can be used as a medicinal product, according to the regulatory requirements (in particular for toxicology), with respect to the benefit provided by the product thus identified.

In the same way, the invention also relates to the methods for optimizing the products which suppress tumor suppression and/or apoptosis, identified using the methods described above, and which make it possible to identify products which can be used as medicinal products.

Thus, the invention also relates to a method for identifying a product having an activity of decreasing and/or inhibiting tumor suppression and/or apoptosis, characterized in that it comprises the steps of:
a) implementing a method according to the invention, for identifying compounds having a certain activity of decreasing tumor suppression and/or apoptosis,
b) modifying the product selected in step a), in particular by grafting residues onto the chemical backbone,
c) testing the product modified in step b) in in vitro and/or in vivo methods, on relevant models of tumor suppression and/or apoptosis,
d) identifying the product which makes it possible to obtain an activity of tumor suppression and/or apoptosis which is decreased compared to the activity obtained for the product selected in step a).

Step d) can also be replaced with a step d′), which would be:
d′) identifying the product which makes it possible to obtain the desired biological effect with less toxicity in an animal model (when one of the models used in step c) is in vivo).

In fact, this also involves being able to obtain the product which exhibits the best (biological activity and clinical effect)/(potential risks for use) ratio.

The parameters to be brought into play in order to obtain these results are all known and within the scope of those skilled in the art who wish to develop novel medicinal products, and can be found, for example, in the directives of the organizations such as the l'Agence du Médicament [French Drug Agency], European Commission or Federal Drug Agency.

The use of the methods according to the present invention requires models which make it possible to determine the binding between TSIP4 and TSIP2, and also an easy measurement of the increases or decrease in the amount of TSIP2 or p53 protein in a eukaryotic cell.

The amount of TSIP2 or p53 protein can be studied by Western Blotting, the revelation being carried out using an antibody directed against said protein. Such antibodies, directed against p53, can in particular be found at Calbiochem, under the reference OP09L.

In order to implement the methods according to the invention requiring studying and screening on mammalian cells, it may be advantageous to overexpress one or other of the TSIP4, TSIP2 and p53 proteins in said cells, two of them, or all three together.

Thus, it is easier to be able to study the variations in the amount of TSIP2 protein or of p53, in particular by comparison of the cells on which the compounds of interest are tested with the same cells, to which the compounds which it is desired to screen are not added.

It is understood that the expression of the two proteins TSIP4 and TSIP2, and p53, can be increased by introducing the genes (in particular the cDNAs) encoding these proteins into the cells, either placed on vectors or by introduction into the chromosome.

When episomal expression is chosen, said mammalian cell is transfected with at least one vector chosen from a vector carrying a DNA fragment encoding TSIP4, a vector carrying a DNA fragment encoding TSIP2, a vector carrying a DNA fragment encoding p53, and a vector carrying a DNA fragment encoding more than one of these proteins. Thus, the same vector can express all the proteins; alternatively, it is possible to introduce several vectors.

It is possible to use vectors which allow expression and easy purification of the TSIP4 and TSIP2 proteins, for example in prokaryotic cells (E. coli, B. subtilis etc.) or eukaryotic cells (yeast such as Saccharomyces, Kluyveromyces, etc.), mammalian cells (HeLa, Cos, Hep-2, etc.) or insect cells (using a Baculovirus system). Thus, it may be advantageous for the proteins to have a tag at their N- or C-terminal end, in order to facilitate the purification. A histidine or GST tag is in particular chosen. These methods are well known to those skilled in the art, who find the suitable plasmids in the catalogues of companies such as Stratagéne.

When the methods according to the invention are implemented on in vitro models, to study the binding between TSIP2 and TSIP4, there are several ways to carry out the procedures.

A protocol which can be used may be as follows:
expression and purification of the TSIP4 and TSIP2 proteins, for example in prokaryotic cells (E. coli, B. subtilis, etc.) or eukaryotic cells (yeast such as Saccharomyces, Kluyveromyces, etc.), mammalian cells (HeLA, Cos, Hep-2, etc.) or insect cells (using a Baculovirus system). It may be advantageous for the proteins to have a tag at their N- or C-terminal end, in order to facilitate the purification. A histidine or GST tag is in particular chosen. These methods are well known to those skilled in the art, who find the suitable plasmids in the catalogues of companies such as Stratagéne;
binding of the proteins to suitable beads. When a GST tag is used, the expressed proteins are bound to sepharose beads exhibiting glutathione;
preparation of proteins by in vitro translation. This can be easily carried out using commercially available vectors (for example available from Promega), which make it possible to clone the cDNAs under the control of well-known promoters (T7 or T3) and to use suitable RNA polymerases to produce the RNAs, and then to effect the expression of the proteins in vitro, using the available kits and following the manufacturer's indications;
coprecipitation of the proteins, by adding the proteins obtained by in vitro translation to the sepharose-glutathione beads to which the proteins from fusion with GST are attached. After a sufficient amount of contact time, the beads are washed and an analysis is carried out by SDS-PAGE gel electrophoresis and autoradiography. The appearance of the bands corresponding to the two proteins clearly shows binding between them.

The use of suitable controls thus makes it possible to define the decrease and/or the inhibition of the binding between TSIP4 and TSIP2, by comparison of the amounts of proteins released after adding the compound tested during the coprecipitation step, with the amounts of proteins released in the controls.

It is also possible to study the binding of the proteins using the FRET (Fluorescence Resonance Energy Transfer) system, which consists in labeling each one of the proteins with a suitable residue, the binding of the two proteins inducing a reaction between each one of the two residues and the emission of a readily detectable fluorescence.

A subject of the present invention is also the compounds which can be obtained using a method according to the invention, in particular the compounds which have an activity of increasing tumor suppression and/or apoptosis, those which have an activity of inhibiting the TSIP4-TSIP2 binding, and also those which have an activity of decreasing and/or inhibiting tumor suppression and/or apoptosis.

The present invention also relates to the peptide sequences corresponding to a region of TSIP4 which interacts with the TSIP2 protein, which can in particular be identified using a method according to the invention.

The invention also relates to the peptide sequences corresponding to a region of TSIP2 which interacts with the TSIP4 protein, which can in particular be identified using a method according to the invention, the method which makes it possible to identify the peptide sequences of TSIP4 which interact with TSIP2 possibly being adapted to determine the peptide sequences of TSIP2 which interact with TSIP4, in particular by adapting the in vitro protocol developed above.

The invention also relates to the nucleotide sequences encoding the peptide sequences thus identified.

It is clear that the term “peptide sequence” or “nucleic acid sequence” or “nucleotide sequence” (the latter two terms being used indifferently) represents sequences which are isolated, i.e. outside of their natural state, and which can in particular be modified by replacing their base units with unnatural units, or by modifying the bonds between the base units (for example phosphorothioates (nucleic acid) or peptide nucleic acids).

An object of the invention is to make it possible to identify compounds which interfere with the TSIP4 and TSIP2 binding, some of these compounds possibly in particular inducing effects on the p53 cascade. Thus, the compounds according to the invention, the peptide sequences according to the invention or the nucleotide sequences according to the invention, as a medicinal product, are also subjects of the invention.

A compound identified using a method according to the invention may be a compound which has a chemical structure, a lipid, a sugar, a protein, a peptide, a protein-lipid, protein-sugar, peptide-lipid or peptide-sugar hybrid compound, or a protein or a peptide to which chemical branches have been added.

Among the chemical compounds envisioned, they may contain one or more (in particular two or three) rings, which may or may not be aromatic, having from three to eight carbon atoms, and also several residues of any type (in particular lower alkyl, i.e. exhibiting between one to six carbon atoms).

These compounds, nucleic acid sequences and peptide sequences can thus be used to prepare a medicinal product intended in particular for the treatment of cancer or of a neurodegenerative disease, depending on the pro- or anti-apoptosis/tumor reversion effect.

The inventors of the present application have, for the first time, demonstrated the fact that the TSIP4 protein binds to the TSIP2 protein. Thus, the present invention also relates to a complex consisting of a TSIP4 protein and a TSIP2 protein.

The present invention also relates to a method for inhibiting the binding of TSIP4 to TSIP2 in a cell, comprising the step of:
a) bringing said cell into contact with a compound identified using a method according to the invention, which inhibits the TSIP4-TSIP2 binding.

The compound thus envisioned can also be a “decoy” peptide derived from the TSIP4 protein or from the TSIP2 protein. The method can be implemented in vitro or in vivo.

The present invention is also directed toward a method for the treatment of a cancer, characterized in that a compound which has been identified according to the present invention and which increases apoptosis and/or tumor reversion is administered to a patient.

A method for the treatment of a neurodegenerative disease, consisting in administering, to a patient, a compound according to the present invention which decreases or inhibits apoptosis, is also a subject of the present invention.

The following examples make it possible to understand more clearly the advantages of the invention and should not be considered as limiting the scope of the invention.

### DESCRIPTION OF THE FIGURES

FIG. 1: description of the TSIP4 mRNA in various tissues.

FIG. 2: differential expression of TSIP 4 in the K562/KS system.

### EXAMPLES

### Example 1

### Binding of TSIP4 and TSIP2

The binding existing between TSIP 4 and TSIP 2 was observed in a double-hybrid system derived from the system developed by Finley and Brent (Interaction trap cloning with yeast, 169-203, in DNA Cloning, Expression Systems: a practical Approach, 1995, Oxford Universal Press, Oxford), using presenilin 1 (TSIP2) as bait and TSIP4 as prey.

The TSIP2 gene (cDNA) was cloned into the plasmid pEG202 known to those skilled in the art for such an application (promoter 67-1511, lexA 1538-2227, ADH Ter 2209-2522, pBR remnants 2540-2889, 2μ ori 2890-4785, YSCNFLP 4923-5729, HIS3 7190-5699, TYIB 7243-7707, RAF_part 7635-7976, backbone pBR 7995-10166, bla 8131-8988).

The TSIP4 gene (cDNA) was cloned into the plasmid pJG4-5, also well known to those skilled in the art (promoter GAL 1-528, fusion cassette 528-849, ADH Ter 867-1315, 2μ ori 1371-3365, TRP1 3365-4250, backbone pUC 4264-6422, Ap 4412-5274).

The reporter plasmid pSH18-34, also known to those skilled in the art, is also used. This plasmid is in particular available from Invitrogen, under the reference number V611-20, and is also already transformed into the strain EGY48 (also called RFY 231), from the same supplier (reference strain alone: C835-00, transformed with pSH18-34: C836-00).

The binding was demonstrated in the yeast strain RFY 231 (described in Finley Jr, et al, 1998, Proc Natl Acad Sci USA, 95 14266-71). This yeast strain has the genotype (MATα trplΔ::hisG his3 ura3-1 leu2::3Lexop-LEU2), and is derived from EGY48 (Guris et al., 1993, Cell, 75, 791-803).

The reporter gene was the LacZ gene.

The study is carried out on a medium containing galactose and not containing leucine, and the presence of colored colonies on these dishes is studied.

It was thus possible to show that the TSIP21 protein binds to the TSIP4 protein, and that the binding is on the 204 C-terminal amino acids of the TSIP2 protein.

### Example 2

### Analysis of the TSIP4 Protein

It appears that the TSIP 4 protein has two alternative forms, one corresponding to SEQ ID No. 2, encoded by SEQ ID No. 1, the other corresponding to amino acids 198-400 of SEQ ID No. 2, encoded by bases 592 to 1200 of SEQ ID No. 1. A putative signal peptide corresponds to amino acids 198-243 of SEQ ID No. 2 (bases 592 to 729 of SEQ ID No. 1).

The sequences described above are also subjects of the invention.

The (complete) TSIP4 protein has 4 predicted transmembrane domains (amino acids 224-244, 260-280, 320-340, and 350-370), the N- and C-terminal ends being located in the cytoplasm. These transmembrane domains, and also the intra- or extracellular domains, are also subjects of the invention, as are the sequences which encode them.

### Example 3

### Expression of the TSIP 4 Protein

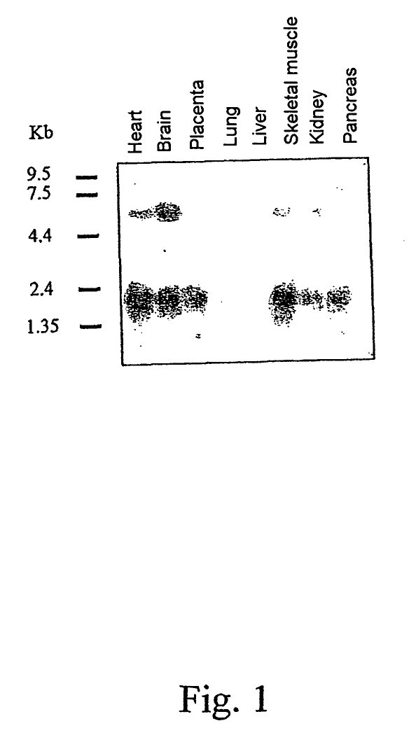

Hybridization of a Northern Blot of various human tissues with a probe corresponding to a partial sequence of TSIP4 shows the existence of two transcripts (approximately 1.8 and 6 kb). The mRNA is found mainly in the heart, the skeletal muscle and the brain. It is also present, to a lesser extent, in the placenta, the pancreas and the kidney. Its expression is very low in the lung and virtually nonexistent in the liver (FIG. 1).

### Example 4

### Differential Expression of TSIP4 in the K562/KS System

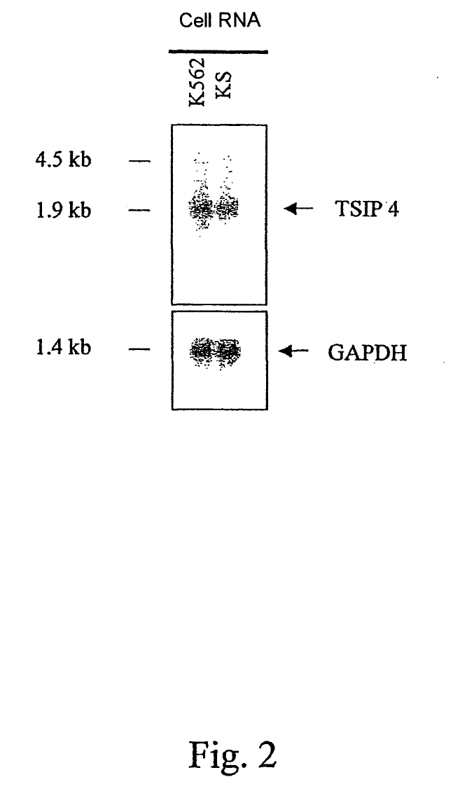

Hybridization of a Northern Blot with the TSIP4 probe showed a decreased signal compared to the K562 line, whereas an equivalent signal is obtained with the control GAPDH probe (FIG. 2). The K562/KS model was described by Telerman et al. (1993, Proc. Natl. Acad. Sci. USA, 90, 8702-6).

The TSIP4 protein is therefore suppressed in a model of tumor suppression.

### Example 5

### Expression of GST Fusion proteins

To obtain GST fusion proteins, the protocol below can be followed:

Preparation of a preculture from an isolated colony of B121 (DE3), transformed with plasmid pGEX-P-1-TSIP4 or pGEX-P-1-TSIP2, in an SB medium with 100 μg/ml of ampicillin, at 37° C.

The plasmids are available from Amersham Pharmacia Biotech AB.

The proteins are the human proteins, encoded by the complementary cDNAs corresponding to the sequences SEQ ID No. 1 (TSIP4) and the complete sequence of TSIP2 (GenBank U50957).

The following day, 250 ml of SB+Amp are inoculated with 5 ml of the preculture.

Growth is at 28° C. or 37° C. depending on the toxicity of the proteins for the host bacteria, until an optical density of between 0.5 and 0.7 is reached.

0.1 mM IPTG is added to induce the protein synthesis.

Growth is at 28° C. or 37° C. for 1 h or 1 h 30.

Centrifugation is carried out at 3000 rpm for 10 min (1800 g, 4° C.).

The precipitate is resuspended in 10 ml of buffer A NP40 (1% of NP40; 10 mM Tris, pH 7.4; 150 mM NaCl; 1 mM EDTA; 10% glycerol; 1 mM DTT; 2 pg/ml aprotinin; 2 μg/ml leupeptin; 2 μg/ml pepstatin; 1 mM AEBSF).

Sonication is carried out 3 times for 15 s at power 50, on ice.

Centrifugation is carried out at 12000 rpm for 10 min (18000 g, 4° C.).

The supernatant is kept at −80° C.

### Example 6

### Protocol to be Followed for Binding of the Fusion Proteins to the Sepharose-Glutathione Beads

2 ml of supernatant are added to 200 μl of beads (prepared after 3 rinses in PBS, 1 rinse in the buffer A NP40, resuspension at 50% (weight by volume) in the buffer A NP40, centrifugation at 3000 rpm each time).

The glutathione-sepharose 4B beads are available from Amersham Pharmacia Biotech AB, under the number 17.0756.01.

Gentle mixing is carried out for at least 1 hour at 4° C.

The beads are rinsed 3 times in the buffer A NP40 without protease inhibitor.

The beads are resuspended in 1 ml of buffer A NP40 with protease inhibitor.

For the analysis by SDS-PAGE electrophoresis, a 20 to 40 μl sample of the resuspended beads is taken and centrifuged for 5 min, the supernatant is discarded, the beads are resuspended in 10 μl of loading buffer X, and heating is carried out at 97° C. for 7 min. The gel is loaded and analyzed after revelation with Coomassie blue, in order to standardize the amount of the fusion proteins to be used.

### Example 7

### Protocol for the in vitro Protein Translation

The TNT Coupled Reticulocyte Lysate System kit from Promega is used with the T7 or T3 RNA polymerases, depending on the vector used to translate and express the proteins (T7 for AIPI and TSIP4 1, T3 for TSIP2) The kit is used according to the manufacturer's instructions (reference L4610).

The proteins incorporate S³⁵-methionine (Amersham Pharmacia).

The products obtained in vitro are analyzed by SDS-PAGE electrophoresis.

After electrophoresis, the gel is placed in a fixing buffer (5% of methanol, 15% of acetic acid, 80% of water) for half an hour and the signal is amplified by immersing the gel in the Amplify product from Amersham Pharmacia (Ref: NAMP100).

A Kodak Biomax MR film is then exposed on the dried gel for a period ranging from one hour to one week, and then developed.

### Example 8

### Protocol for the Coprecipitation of the Proteins

30 μl of the sepharose-glutathione beads coupled to the GST fusion proteins, after standardization of the amounts, are rinsed in buffer B (1% NP40, 50 mM Tris-HCl, 150 mM NaCl, 2 μl/ml leupeptin, 1% aprotinin, 1 mM ABESF).

5 to 10 μl of the in vitro translation product as obtained in Example 3 are then added, depending on the amount of the product observed by autoradiography.

After contact overnight, the beads are rinsed 10 times with buffer A NP 40, without antiproteases.

Analysis is carried out by SDS-PAGE and autoradiography.

It is thus possible to show the binding between the TSIP4 and TSIP2 proteins.

### Example 9

### Protocol for Screening Compounds Which Interfere with the TSIP4-TSIP2 Binding

The assays described in Examples 5 to 8 are carried out, adding the compounds which it is desired study to the step of Example 8, and comparison is made with the results obtained when the compounds are not added.

### References

Buckholz, (1993), Curr. Op. Biotechnology 4, 538.
Carter, (1993) Curr. Op. Biotechnology 3, 533.
Duck et al. (1990), Biotechniques, 9, 142.
Edwards et Aruffo (1993), Curr. Op. Biotechnology 4, 558.
Epstein (1992) Medecine/Sciences, 8, 902.
Guatelli et al. (1990), Proc. Natl. Acad. Sci. USA 87: 1874.
Kievitis et al. (1991), J. Virol. Methods, 35, 273.
Köhler et Milstein. (1975) Nature 256, 495.
Kwoh, et al. (1989), Proc. Natl. Acad. Sci. USA, 86, 1173.
Landegren et al. (1988) Science 241, 1077.
Luckow (1993), Curr. Op. Biotechnology 4, 564.
Matthews et al. (1988), Anal. Biochem., 169, 1-25.
Miele et al. (1983), J. Mol. Biol., 171, 281.
Neddleman et Wunsch (1970) J. Mol. Biol. 48: 443
Olins et Lee (1993), Curr. Op. Biotechnology 4: 520.
Perricaudet et al. (1992). La Recherche 23: 471.
Pearson et Lipman (1988) Proc. Natl. Acad. Sci. USA 85: 2444
Rohlmann et al. (1996) Nature Biotech. 14: 1562.
Rolfs, A. et al. (1991), Berlin: Springer-Verlag.
Sambrook et al. (1989) Molecular cloning: a laboratory manual. 2nd Ed. Cold Spring Harbor Lab., Cold Spring Harbor, New York.
Segev, (1992), Kessler C. Springer Verlag, Berlin, New-York, 197-205.
Smith et Waterman (1981) Ad. App. Math. 2: 482
Stewart et Yound (1984), Solid phase peptides synthesis, Pierce Chem. Company, Rockford, 111, 2ème éd., (1984).
Temin, (1986) Retrovirus vectors for gene transfer. In Kucherlapati R., ed. Gene Transfer, New York, Plenum Press, 149-187.
Walker (1992), nucleic acids res. 20: 1691.

1. A purified or isolated nucleic acid, characterized in that it comprises a nucleic acid sequence chosen from the group of following sequences:
a) SEQ ID No. 1;
b) a nucleic acid sequence exhibiting a percentage identity of at least 80%, after optimal alignment, with a sequence defined in a);
c) a nucleic acid sequence which hybridizes, under high stingency conditions, with a nucleic acid sequence defined in a);
d) the complementary sequence or the RNA sequence corresponding to a sequence as defined in a), b) or c),

2-38. (Canceled)
